(19) United States
(12) Patent Application Publication
(10) Pub. No.: US 2021/0374122 A1
(43) Pub. Date: Dec. 2, 2021

(54) METHOD AND SYSTEMS FOR CLEANING AND ENRICHING DATA FROM A REAL-TIME LOCATING SYSTEM

(71) Applicant: KONINKLIJKE PHILIPS N.V., EINDHOVEN (NL)

(72) Inventors: Supriyo Chatterjea, Eindhoven (NL); Alexander Furnica, Eindhoven (NL); Evert Jan van Loenen, Waalre (NL); Erik Bresch, Eindhoven (NL)

(21) Appl. No.: 17/146,579

(22) Filed: Jan. 12, 2021

Related U.S. Application Data
(60) Provisional application No. 63/030,331, filed on May 27, 2020.

(51) Int. Cl. G06F 16/23; H04W 4/029; G16H 40/20

(52) U.S. Cl. CPC G06F 16/2365; G16H 40/20; H04W 4/029

(57) ABSTRACT

A method for modifying data from a Real-Time Location System (RTLS) involving: receiving building arrangement information having two locations and one or more identifiable characteristics of the two locations; obtaining identifying information of at least one tag and at least two sensors in the RTLS; generating a mapping of the identifying information with the one or more identifiable characteristics of the two locations; receiving location information of the at least one tag based on the mapping, the location information indicating a presence of the at least one tag in the two locations at separate times; determining that the location information includes at least one gap because the at least one tag cannot proceed directly between the two locations in the building arrangement information; and updating the location information based on an interpolation of the location information to fill in the at least one gap.

### FIELD OF THE DISCLOSURE

The present disclosure is directed generally to methods and systems for cleaning and enriching data from a real-time locating system. More specifically, the present disclosure is directed generally to methods and systems for filtering out data that adds no value and interpolating missing data from a real-time locating system.

### BACKGROUND

As populations around the world age and the number of admitted patients increases, hospitals are increasingly under pressure to make better use of resources which include staff, equipment, and medical facilities. Effective planning of resources is crucial for improving hospital productivity and quality and containing or reducing costs. Measuring and analyzing existing workflows provide hospital management a better understanding of how efficiently various departments function. Such analyses also provide the opportunity to identify ways to optimize the workflows.

In order to acquire accurate representations of workflows and patient care pathways, the following criteria must be met. First, every patient, staff member, and asset must be tracked at all times. Second, the spatial resolution (e.g., room level, bed level, etc.) must be high enough to provide the appropriate level of information needed to accurately track and characterize hospital workflows. Third, no data stream is ever interrupted or missing. Unfortunately, the above conditions are rarely all present, resulting in incomplete data that, on its own and in an unmodified state, does little to represent reality. To complete the data, the missing data would have to be filled in manually based on realistic room transitions and removing unknown reports. Yet, such an endeavor is not realistic in a hospital setting since the data is updated in real-time (e.g., every tracked tag reports every few seconds). Manual systems and methods cannot keep up with the incoming data.

### SUMMARY OF THE DISCLOSURE

The present disclosure is directed to inventive methods and systems for systematically cleaning and enriching data from a Real-Time Location System (RTLS). Generally, embodiments of the present disclosure are directed to improved methods and systems for transforming raw RTLS data so that it is easier to analyze. Applicant has recognized and appreciated that it would be beneficial to provide methods and systems for systematically cleaning and enriching RTLS data by removing irrelevant observations and interpolating missing location data based on floor plan data. Various embodiments and implementations herein are directed to applying location mapping file(s) based on floor plan data and an interpolating module to raw RTLS data. The methods also filter out unused information, distinguish between tag roles and floors of a facility, and define valid movement patterns based on time-related conditions. The methods and systems described herein advantageously extract optimal value out of an RTLS regardless of the context.

Real-Time Location Systems (RTLS) enable the tracking and management of medical equipment, staff, and patients in real-time. RTLS systems enable patient care facilities to capture workflow efficiencies, reduce costs, and increase clinical quality. Generally, RTLS systems include tags and/or badges, platforms (Wi-Fi, Infrared, Ultrasound, and others), readers or other corresponding hardware infrastructure, and other components (servers, middleware & end-user software). While RTLS data, in its raw form, fulfils its original purpose of providing location data, the lack of quality in the data makes it difficult to extract meaningful insights from the data. RTLS data presents the possibility of calculating elapsed times between locations (and therefore tasks), and mapping movement and workflow patterns to floor plans just to name a few. However, due to missing data that happens naturally by, for example, covering a patient tag with a blanket, or due to packet loss over the radio channel, it becomes difficult to extract these insights.

Generally, in one aspect a computer-based method for modifying data from a Real-Time Location System (RTLS) is provided. The method may include the step of receiving building arrangement information, wherein the building arrangement information comprises at least two locations and one or more identifiable characteristics of the at least two locations and the step of obtaining identifying information of at least one tag and at least two sensors in the RTLS.

The method may further include the step of generating a mapping of the identifying information with the one or more identifiable characteristics of the at least two locations and the step of receiving location information of the at least one tag based on the mapping. The location information indicates a presence of the at least one tag in the at least two locations at separate times.

The method may further include the step of determining that the location information includes at least one gap because the at least one tag cannot proceed directly between the at least two locations in the building arrangement information and the step of updating the location information based on an interpolation of the location information to fill in the at least one gap.

According to an embodiment, the one or more identifiable characteristics comprise unique names of the at least two locations or x and y coordinates of the at least two locations in the building arrangement information.

According to an embodiment, the one or more identifiable characteristics comprise information identifying areas neighboring the at least two locations. In a further embodiment, the information identifying areas neighboring the at least two locations comprises names or x and y coordinates.

According to an embodiment, the step of updating the location information is achieved by a shortest path algorithm.

According to an embodiment, the identifying information comprises a role associated with the tag and the interpolation is based at least in part on the role associated with the tag.

According to an embodiment, the interpolation is based at least in part on at least one weighting factor indicative of a transition being more or less likely. In a further embodiment, the weighting factor is based at least in part on a time of day.

According to an embodiment, the method may further include the step of detecting a recurrence of two signals of a minimum length within a predetermined time interval having a start time and an end time. In a further embodiment, the method may further include the step of overwriting at least one identifier of a signal of the two signals occurring after the start time and before the end time with “0”.

According to an embodiment, the at least two locations are arranged on separate floors of the building arrangement information.

Generally, in another aspect, a system for modifying data of a Real-Time Location System (RTLS) is provided. The system can include building arrangement information comprising at least two locations and one or more identifiable characteristics of the at least two locations and a processor. The processor of the system can be configured to: receive the building arrangement information; obtain identifying information of at least one tag and at least two sensors in the RTLS; receive a mapping of the identifying information with the one or more identifiable characteristics of the at least two locations; receive location information of the at least one tag based on the mapping, the location information indicating a presence of the at least one tag in the at least two locations at separate times; determine that the location information includes at least one gap because the at least one tag cannot proceed directly between the at least two locations in the building arrangement information; and update the location information based on an interpolation of the location information to fill in the at least one gap.

According to an embodiment, the at least two locations are arranged on separate floors of the building arrangement information.

According to an embodiment, the one or more identifiable characteristics comprise unique names of the at least two locations or x and y coordinates of the at least two locations in the building arrangement information.

According to an embodiment, the one or more identifiable characteristics comprise information identifying areas neighboring the at least two locations.

In various implementations, the processor described herein may take any suitable form, such as, one or more processors or microcontrollers, circuitry, one or more controllers, a field programmable gate array (FGPA), or an application-specific integrated circuit (ASIC) configured to execute software instructions. Memory associated with the processor may take any suitable form or forms, including a volatile memory, such as random-access memory (RAM), static random-access memory (SRAM), or dynamic random-access memory (DRAM), or non-volatile memory such as read only memory (ROM), flash memory, a hard disk drive (HDD), a solid-state drive (SSD), or other non-transitory machine-readable storage media. The term “non-transitory” means excluding transitory signals but does not further limit the forms of possible storage. In some implementations, the storage media may be encoded with one or more programs that, when executed on one or more processors and/or controllers, perform at least some of the functions discussed herein. It will be apparent that, in embodiments where the processor implements one or more of the functions described herein in hardware, the software described as corresponding to such functionality in other embodiments may be omitted. Various storage media may be fixed within a processor or may be transportable, such that the one or more programs stored thereon can be loaded into the processor so as to implement various aspects as discussed herein. Data and software, such as the algorithms or software necessary to analyze the data collected by the tags and sensors, an operating system, firmware, or other application, may be installed in the memory.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a schematic block diagram of an example Real-Time Location System for asset tracking in accordance with aspects of the present disclosure;

FIG. 2 is an exemplary floor plan of a patient care environment for a Real-Time Location System in accordance with aspects of the present disclosure;

FIG. 3 is a schematic diagram of data from a Real-Time Location System without and with enrichment in accordance with aspects of the present disclosure; and

FIG. 4 is an example flowchart showing a process for modifying data from a Real-Time Location System in accordance with aspects of the present disclosure.

### DETAILED DESCRIPTION OF EMBODIMENTS

The present disclosure describes various embodiments of methods and systems for cleaning and enriching data from a Real-Time Location System. More specifically, Applicant has recognized and appreciated that it would be beneficial to effectively transform raw RTLS data into a format that does not contain missing or otherwise misreported location data and includes useful features such as enriched and accurate movement patterns. Advantageously, the converted raw data is more conducive to analysis. Additionally, Applicant has recognized and appreciated that it would be beneficial to provide methods and systems for systematically cleaning and enriching RTLS data by removing irrelevant observations and providing a logical interpolation of location data.

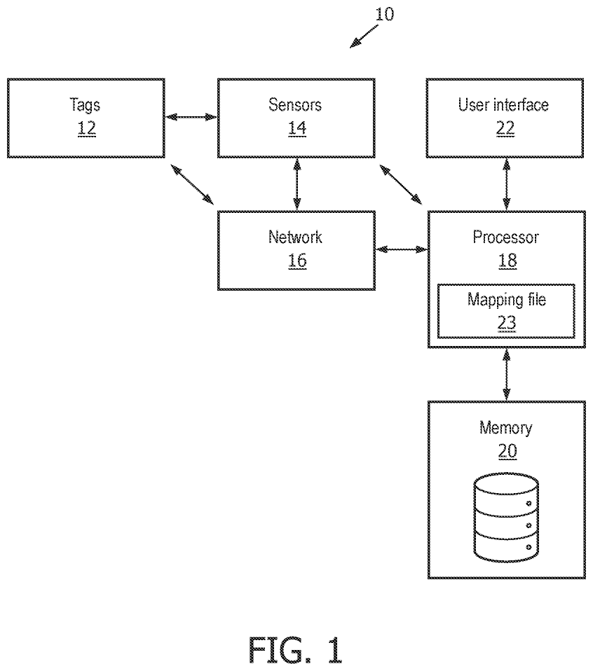

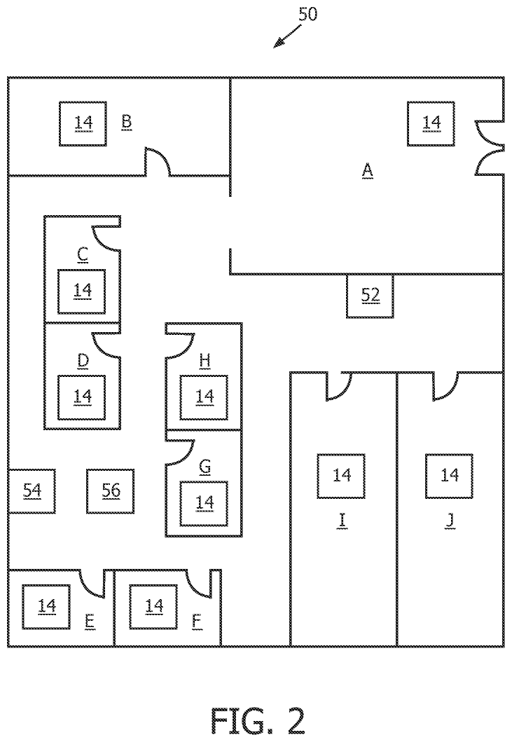

Referring to FIG. 1, a schematic depiction of an asset tracking system 10 is provided. FIG. 2 shows an exemplary floor plan of a patient care environment 50 to be used with asset tracking system 10 in embodiments of the present disclosure. The following should be considered in view of FIGS. 1 and 2. Tracking system 10 applies to any real-time location system that uses any kind of protocol and/or technology, e.g., IR, WiFi, Bluetooth, Ultrasound, RFID etc. Tracking system 10 includes portable identification tags and/or badges 12, specialized fixed position sensors 14 (e.g., readers, receivers, and/or exciters), network 16, computer processor 18, and memory 20. The portable identification tags 12 can be embodied as any suitable identifying tags associated with subjects or objects such as patients, equipment, or staff. For example, the portable identification tags 12 can be embodied as infrared (IR) tags having an IR sensor. The fixed position sensors 14 can be embodied as IR receivers distributed throughout any suitable patient care environment, such as, a hospital, and are configured to continuously transmit data to be received by the portable identification tags 12. In embodiments, the sensors 14 are arranged within areas of the patient care environment 50 and each sensor 14 is configured to transmit wireless signals or beacons with identifying information (e.g., a unique identifier or code) in real-time. The portable identification tags 12 are configured to receive or pick-up the wireless signals from the sensors 12 as they move about the floor plan and transmit them over any suitable radio frequency channel within network 16 to computer processor 18.

Depending on the selected technology, the system 10 locates the tags and therefore the location of the tagged entity. For example, the portable identification tags 12 can be configured to transmit a 900 MHz radio-frequency signal including receiver information and a tag identifier to access points within a wireless network 16 (e.g., a WiFi network). The access points can send the tag identifier and the receiver information to the computer processor 18 via WiFi routers to locate the identification tags 12. In alternate embodiments, the identification tags 12 itself can perform the localization and then transmit the location information to the processor 18. In still other embodiments, the portable identification tags 12 can transmit data over a Bluetooth communication channel employing short-wavelength UHF radio waves and personal area networks. WiFi and Bluetooth technologies can use a received signal strength indication (RSSI), angle of arrival (AoA), and/or time of arrival (ToA) to determine a location of the identification tags 12. It should be appreciated that any other suitable alternate parameters or combination of parameters can be used additionally or alternatively.

In other embodiments, the portable identification tags 12 or the sensors 14 can be configured to additionally or alternatively emit ultrasonic sound waves with frequencies typically above 20 kHz. In these embodiments, the RSSI, AoA, and/or ToA parameters and/or any other suitable parameter(s) can be used to locate an identification tag. For example, time-of-flight of the sound waves can be measured from a tag to one or more sensors and the shortest time-of-flight can indicate which sensor the tag is closest to. In embodiments, the location of the tag can be identified using trilateration techniques. In systems with ultrasonic sensors, any suitable transducer can be configured to send one or more pulses and receive the echo. A distance measurement between the tag and the sensor can be calculated based on time-of-flight (e.g., the time between sending the one or more pulses and receiving the reflected sound signal). The calculation can occur at the sensor or remote from the sensor.

As described herein, the system 10 is not limited to IR tags and IR receivers. In other embodiments, the portable identification tags 12 can be embodied as Bluetooth tags and the sensors 14 can be embodied as fixed mounted Bluetooth receivers where the receivers are continuously scanning for nearby beacons from the tags on the entities. In embodiments, the receivers can transmit detected beacon identifying information and distance to the detected beacon tag to the computer processor 18 via a suitable network 16. Still in other embodiments, the tags could be embodied as radio-frequency identification (RFID) tags, for example, active RFID tags.

Depending on the system, varying degrees of granularity can be achieved. Some RTLS systems can enable tracking in a hospital unit or floor. Other clinical-grade systems can achieve room, bed, bay, and even shelf-level tracking.

Exemplary locations of sensors 14 include entryways of rooms A, B, C, D, E, F, G, H, I, and J in the floor plan of the patient care environment 50, patient beds within the rooms, hand-washing stations 52, storage closets 54, and medical equipment 56 etc. Any of the locations contemplated herein in the patient care environment can include areas that are accessible to patients and staff, staff only, or a subset of patients and/or staff. In example embodiments, the location data can be transmitted between the tags 12 and the sensors 14 and the tags 12 can be configured to transmit the location data via network 16 to computer processor 18 via any suitable wired/wireless communication channels. Alternatively, the sensors 14 can be configured to transmit location data via network 16 to computer processor 18 via any suitable wired/wireless communication channels. In embodiments, the location data can be transmitted directly to computer processor 18 without passing through network 16. The location data can be stored in memory 20 via the wired/wireless communication channels. It should be appreciated that in other embodiments, additional or fewer locations can be included in asset tracking system 10 and in any suitable arrangement.

In embodiments, the floor plan of the patient care environment 50 can be an image provided by a user via user interface 22 associated with the processor 18 in system 10. In alternate embodiments, the floor plan can be retrieved from memory 20 via a system bus or any suitable alternative. User interface 22 may include one or more devices or software for enabling communication with a user. The devices can include a touch screen, a keypad, touch sensitive and/or physical buttons, switches, a keyboard, knobs, levers, a display, speakers, a microphone, one or more indicator lights, audible alarms, a printer, and/or other suitable interface devices. The user interface can be any device or system that allows information to be conveyed and/or received, and may include a graphical display configured to present to a user (e.g., a hospital administrator) views and/or fields configured to receive entry and/or selection of information related to tags and sensors and their associated entities and locations, respectively. The user interface 22 may be located within one or more components of the system (e.g. processor 18) or may be located remote from the system and in communication with the system via wired/wireless communication channels.

The processor 18 is capable of executing instructions stored in memory 20 or otherwise processing data to, for example, perform one or more steps of the methods described herein. Processor 18 may include one or more modules, such as, a cleaning module, an interpolation module, and a hopping removal module as further described below. The term “hopping” as used herein refers to an oscillation-like recurrence of two beacons of a minimum length within a predetermined time interval in the output RTLS data file as further described below. The cleaning, interpolation, and hopping removal modules can be part of the same module or separate modules. It should be appreciated that while the system 10 is shown as including one of each described component, the various components may be duplicated in various embodiments. For example, processor 18 can include multiple microprocessors that are configured independently to execute the methods described herein or are configured to perform steps or subroutines of the methods described herein in cooperation. In embodiments where one or more of the components of the system is implemented in a cloud computing system, the various hardware components may belong to separate physical systems. Any other suitable configurations are possible to carry out the inventive methods and systems described herein.

The tracking system 10 stores raw RTLS data which captures a state of an individual tag at a particular moment in time. This raw RTLS data includes the following information: a timestamp (e.g., the time that the data was transmitted); a tag identifier (e.g., the unique identifier of the tag); sensor identifier (e.g., a unique identifier of the sensor that received the data); button presses (e.g., different indicators informing of whether or not one of four buttons was pressed at the time the data was transmitted); and tag type (e.g., a categorical value determining a model of the tag). Thus, based on the raw RTLS data alone the amount of information that can be gleaned about the state of an individual tag is limited. For example, while we can identify which particular tag received data from which particular sensor at a particular time, it is not known where this particular sensor is located or where it is positioned within an overall floor plan. The location of the particular sensor or its positional data within an overall floor plan can be described as contextual information.

Such contextual information can be provided in the tracking system 10 according to aspects of the present disclosure as described below. For example, the processor 18 of the system 10 can be provided with a mapping file 23 that contains the identifiers of each tag 12 and sensor 14 and additional information. The identifiers of each tag 12 and sensor 14 can be retrieved from software of the tracking system 10. The mapping file 23 is manually created and maps each identifier of each tag and sensor with a name of a location or entity it represents, its x and y coordinates on an image of the floor plan, an identifier of each of its neighboring locations, and the x and y coordinates of all of its doors/transition points to all of its neighboring locations. The mapping file 23 can be generated in any suitable software program, for example, Microsoft Excel.

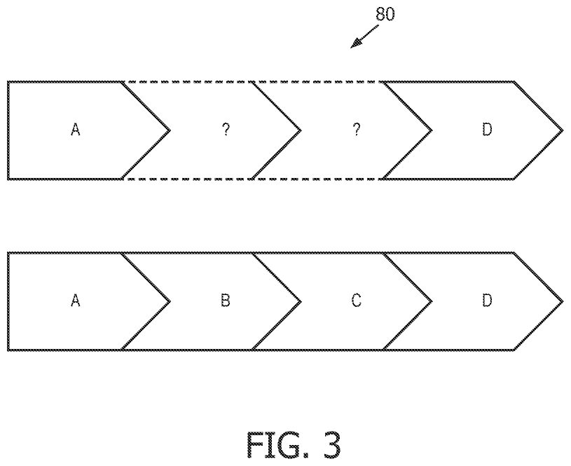

Accurately tracking tags within system 10 can be complicated because tags can pass through or by locations without picking up signals from sensors or transmitting their signal to the appropriate sensor. Such a missing transmission can happen when, for example, the receiver of the tag 12 is occluded (e.g., covered by a blanket) and thus cannot receive data from the sensors 14. Other times data packets can be lost in the radio-frequency interface. Still other times the movement speed of the tag is faster than its update rate. In these scenarios and other similar situations, one or more transitions of an individual tag from one location to one or more other locations can be missed and the data generated by the tracking system can include gaps. The gaps manifest themselves as transitions of an individual tag from one location to another location that is not realistically or logically possible. The top portion of FIG. 3 shows example data 80 from tracking system 10 that includes two such gaps. In other words, the data shows an individual tag transitioning from location A to location D without any data of transitions in between. With reference to the floor plan of the patient care environment 50 of FIG. 2, it is not possible for an individual tag to transition from location A to location D without passing by locations B and C. Thus, the tracking system 10 can include one or more components that interpolate the missing locations so that the data no longer has transition gaps as shown in the bottom portion of FIG. 3.

System 10 can include a cleaning and interpolation module configured to provide the missing locations discussed above. An example interpolation module uses Dijkstra's shortest path algorithm as described below. In a first step of the interpolation module, all locations are marked as unvisited. In other words, a set of all unvisited locations is created and called a first unvisited set.

In a second step of the interpolation module, a first location (e.g., before an identified gap in the data) is designated an initial location and a distance value of zero is assigned to the initial location. The remaining locations in the unvisited set are assigned distance values of infinite.

In a third step of the interpolation module, all of the locations in the unvisited set to which a physical/feasible travel path exists from the initial location are identified and called a second unvisited set. A tentative distance is calculated to each location in the second unvisited set through the initial location. For example, if the initial location A has a distance of 6 and an edge connecting the initial location A with a neighboring location B has a distance of 2, then the distance to location B through location A equals the sum of the initial distance plus the distance of the edge (e.g., 6+2=8). If location B was previously assigned a distance value that is greater than 8, then the interpolation module updates the distance value of location B to have an assigned distance value of 8. In situations where location B was previously assigned a distance value that is smaller than 8, then the interpolation module keeps the smaller assigned distance value without updating the distance value.

Once all of the locations in the second unvisited set have tentative distances calculated and updated where applicable, the initial location is marked as visited and removed from the set of unvisited locations in a fourth step of the interpolation module. After a location is marked as visited in the interpolation module it is not checked again.

In a fifth step, the interpolation module determines whether the destination location has been marked visited (e.g., when planning a route between two specific locations) or if the smallest tentative distance among the locations in the unvisited set is infinity (e.g., when planning a complete traversal; occurs when there is no physical/feasible travel path between the initial location and remaining unvisited locations). If either scenario occurs, the interpolation module performs no further processing. The algorithm has finished.

If neither of the scenarios described above in the fifth step occurs, then the interpolation module identifies the location in the unvisited set with the smallest tentative distance and assigns that location as the new initial location. The interpolation module then proceeds again with the third step of the interpolation module where all of the locations in the unvisited set to which a physical/feasible travel path exists from the newly assigned initial location are identified and called a third unvisited set. A tentative distance is calculated to each location in the third unvisited set through the newly assigned initial location. The process continues to the fourth and fifth steps described above.

In embodiments using the room transition matrix described above, some interpolated paths might not be likely or possible since one or more areas of a patient care environment might be accessible only to staff or employees and not at all available to patients. One or more areas of a patient care environment might only be accessible to a limited subset of the staff or employees and not at all available to patients. Based on the room transition matrix described above, a patient path may be incorrectly interpolated to contain a staff-only corridor. Thus, embodiments of the tracking system 10 can also account for the role of the particular tag being tracked to improve the accuracy of the data. To achieve this functionality, the processor 18 of the system 10 can be provided with separate mapping files (e.g., room transition matrixes) depending on the role associated with the tag. The additional information mentioned above can include tag role (e.g., patient or staff) in embodiments of the present disclosure. The cleaning and interpolation module of the processor 18 is configured to initially identify the tag role, if available, and select the appropriate mapping file or room transition matrix to be used in the shortest path algorithm described above. Limiting the available transitions based on tag role can have the effect of eliminating certain neighboring locations and/or x and y coordinates of some doors/transition points.

In embodiments of the tracking system 10 described herein, the processor 18 of the system 10 can be provided with a time-limited transition matrix to further enhance the accuracy of the data. One or more transitions of an individual tag from one location to another may be valid or possible or likely depending on certain predefined conditions which are only valid at particular times. For example, doors connecting two corridors may be opened/closed depending on the time of the day. Thus, valid tag transitions for the tracking system 10 can be based on the time-limited transition matrix provided to the processor 18. Additionally or alternatively, some (soft) prior information can be provided in the interpolation module of the processor (i.e., some weighting factors of prior belief that some trajectories through certain rooms are more likely than others (for a given tag-role at a given time of the day).

Embodiments of the tracking system 10 can also include a hopping removal module in processor 18. The term “hopping” as used herein refers to an oscillation-like recurrence of two beacons of a minimum length within a predetermined time interval in the output RTLS data file. An example of hopping is observed in an output RTLS data file where two beacons (e.g., “A” and “B”) recur in an oscillation-like manner where the minimum length is “ABAB” and the predetermining time interval is within 10 seconds. The predetermined time interval can also be shorter than 10 seconds. Hopping can occur at regions bordering two sensors where the two sensors receive back-to-back signals from an identification tag within a short period of time or when an IR sensor of a tag receives back-to-back signals from two sensors. When hopping is detected, the reported beacon identifiers in between (but excluding) the start and end times are overwritten with “0” in the output RTLS data file.

While the transitions described above are based on an individual tag moving along the x and y axes of a single floor of a patient care environment, it should be appreciated that embodiments can also take into account transitions between multiple floors of a patient care environment. Example embodiments can account for transitions between floors by defining a constant z-axis value to represent a consistent distance between floors and assigning each room or location a z value based on its floor as shown in the following mathematical equation:

i_{z}=Z*Floor

where i_{z} is the Z-value of the room i, Z is the constant floor distance, and Floor is the floor that room i is currently on.

If a visualization is in two dimensions, then floors can be overlaid and centered on their main transition point, i.e., the elevators. When a transition to a new floor occurs, the background image of the floor plan is switched to the new floor. If the visualization desired is in three dimensions, then the z-axis becomes useful to differentiate between rooms on different floors. Thus, using the approach described above to account for multiple floors allows for flexibility regarding future visualizations advantageously.

As described above, z can represent a constant floor distance in embodiments of the present disclosure. In additional embodiments, z can represent the time elapsed to make the transition between the floors at different times of the day. This is advantageous if there were different congestion patterns for elevators, for example.

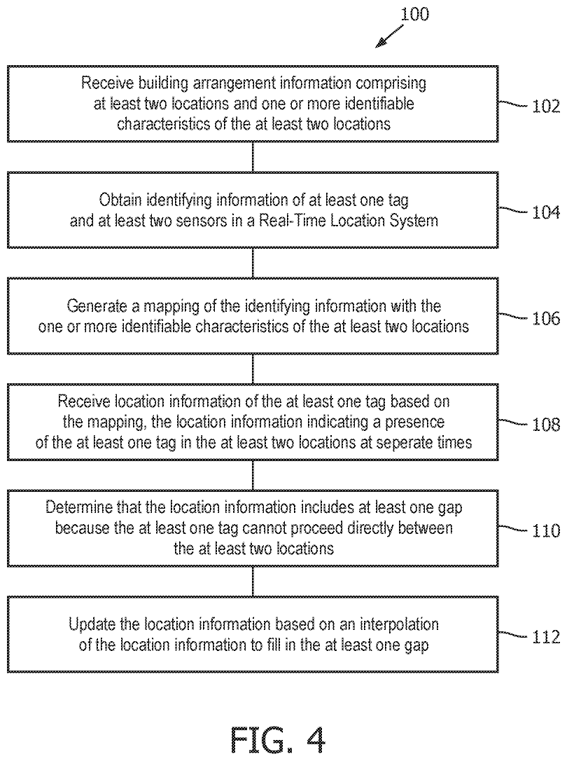

Referring to FIG. 4, a computer-based method 100 for modifying data from a Real-Time Location System (RTLS) is provided. The method 100 includes the step of receiving 102 building arrangement information (e.g., floor plan information) including at least two locations and one or more identifiable characteristics of the at least two locations. The building arrangement information can be provided by a user via the user interface 22 and stored in memory 20 thus, processor 18 can be configured to retrieve the building arrangement information from memory 20. The at least two locations can be any of the locations described herein (e.g., A, B, C, D, E, F, G, H, I, J, 52, 54, and 56). The one or more identifiable characteristics can include unique names or other labels identifying the locations or any other identifying features described herein.

The method further includes the step of obtaining 104 identifying information of at least one tag and at least two sensors in a Real-Time Location System. The identifying information can include the timestamp, tag identifier, sensor identifier, button presses, and/or tag type or any other suitable information as contemplated within the present disclosure.

The method further includes the step of generating 106 a mapping of the identifying information with the one or more identifiable characteristics of the at least two locations. The mapping can be embodied as mapping file 23 described herein or any suitable alternative.

The method further includes the step of receiving 108 location information of the at least one tag based on the mapping generated. The location information indicates a presence of the at least one tag in the at least two locations at separate times.

At step 110 in FIG. 4, the method includes determining that the location information includes at least one gap because the at least one tag cannot proceed directly between the at least two locations in the building arrangement information.

The method lastly includes updating 112 the location information based on an interpolation of the location to fill in the at least one gap. The interpolation module described herein is one example method of carrying out the updating of the location information.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A computer-based method for modifying data from a Real-Time Location System (RTLS), the method comprising the steps of:
receiving building arrangement information, wherein the building arrangement information comprises at least two locations and one or more identifiable characteristics of the at least two locations;
obtaining identifying information of at least one tag and at least two sensors in the RTLS;
generating a mapping of the identifying information with the one or more identifiable characteristics of the at least two locations;
receiving location information of the at least one tag based on the mapping, the location information indicating a presence of the at least one tag in the at least two locations at separate times;
determining that the location information includes at least one gap because the at least one tag cannot proceed directly between the at least two locations in the building arrangement information; and
updating the location information based on an interpolation of the location information to fill in the at least one gap.

2. The method of claim 1, wherein the one or more identifiable characteristics comprise unique names of the at least two locations or x and y coordinates of the at least two locations in the building arrangement information.

3. The method of claim 1, wherein the one or more identifiable characteristics comprise information identifying areas neighboring the at least two locations.

4. The method of claim 3, wherein the information identifying areas neighboring the at least two locations comprises names or x and y coordinates.

5. The method of claim 1, wherein the step of updating the location information is achieved by a shortest path algorithm.

6. The method of claim 1, wherein the identifying information comprises a role associated with the tag and the interpolation is based at least in part on the role associated with the tag.

7. The method of claim 1, wherein the interpolation is based at least in part on at least one weighting factor indicative of a transition being more or less likely.

8. The method of claim 7, wherein the weighting factor is based at least in part on a time of day.

9. The method of claim 1, further comprising detecting a recurrence of two signals of a minimum length within a predetermined time interval having a start time and an end time.

10. The method of claim 9, further comprising overwriting at least one identifier of a signal of the two signals occurring after the start time and before the end time with “0”.

11. The method of claim 1, wherein the at least two locations are arranged on separate floors of the building arrangement information.

12. A system for modifying data of a Real-Time Location System (RTLS), comprising:
building arrangement information, wherein the building arrangement information comprises at least two locations and one or more identifiable characteristics of the at least two locations; and
a processor configured to:
receive the building arrangement information;
obtain identifying information of at least one tag and at least two sensors in the RTLS;
receive a mapping of the identifying information with the one or more identifiable characteristics of the at least two locations;
receive location information of the at least one tag based on the mapping, the location information indicating a presence of the at least one tag in the at least two locations at separate times;
determine that the location information includes at least one gap because the at least one tag cannot proceed directly between the at least two locations in the building arrangement information; and
update the location information based on an interpolation of the location information to fill in the at least one gap.

13. The system of claim 12, wherein the at least two locations are arranged on separate floors of the building arrangement information.

14. The system of claim 12, wherein the one or more identifiable characteristics comprise unique names of the at least two locations or x and y coordinates of the at least two locations in the building arrangement information.

15. The system of claim 12, wherein the one or more identifiable characteristics comprise information identifying areas neighboring the at least two locations.
